(19) United States
(12) Patent Application Publication
(10) Pub. No.: US 2003/0068308 A1
(43) Pub. Date: Apr. 10, 2003

(54) Intrasplenic encapsulated cell therapy

(76) Inventors: Jacek Rozga, Westlake Village, CA (US); Achilles Demetriou, Los Angeles, CA (US)

Correspondence Address: Seth D. Levy, Esq., Pillsbury Winthrop LLP, Suite 2800, 725 South Figueroa Street, Los Angeles, CA, 90017-5406, US

(21) Appl. No.: 10/132,443

(22) Filed: Apr. 25, 2002

Related U.S. Application Data
(60) Provisional application No. 60/328,385, filed on Oct. 9, 2001.

(51) Int. Cl. A61K 48/00; C12N 5/08

(52) U.S. Cl. 424/93.21; 424/93.7; 435/366

(57) ABSTRACT

Disclosed herein are a system and method of implanting encapsulated cells in a recipient's body. Cells are implanted in the spleen of a recipient, thereby allowing the encapsulated cells to survive without suffering immediate/early cell anoxia, inadequate cell nourishment, cell death, or immune rejection. Moreover, immunosuppression is reduced or even not required, and oxygen and various nutrients may be provided to the cells without need for artificially prompting additional vascularization. Furthermore, as the implantation or transplantation may be performed percutaneously or endoscopically (laparoscopically), or by a similar minimally invasive means, a recipient need not endure a highly traumatic surgical procedure.

### RELATED APPLICATIONS

This application claims the benefit of priority under 35 U.S.C. §119 of provisional U.S. Application Serial No. 60/328,385, filed Oct. 9, 2001, the contents of which are hereby incorporated by reference.

### FIELD OF THE INVENTION

Embodiments of the present invention are directed to the implantation of encapsulated cells, and, in particular, to the implantation of encapsulated cells in the spleen of a recipient.

### BACKGROUND OF THE INVENTION

For many diseases, cell therapy—implanting living cells within the body—could be a simple, low risk, and cost-effective alternative to whole organ replacement. It could also allow efficient use of donor organs, which are in critically short supply. For some patients, whole organ transplantation is not even an option. Therapeutic agents could also be delivered to patients by implanting cells which produce such therapeutic agents. For example, if one could successfully transplant insulin producing cells (e.g, pancreatic islet cells, insulinoma cells, isolated pancreatic beta cells) in diabetic patients, one might provide a complete alternative to insulin therapy.

Cell therapy using autologous (self) or mismatched (allogeneic, xenogeneic) cells is likely to succeed clinically only if cells survive at the transplantation site and are protected against immune rejection. Many cell transplantation techniques have been introduced over the years to achieve these objectives, only to be discarded after more careful evaluation and data analysis. In searching for optimal localization, cells have been introduced virtually into every body site including the liver, spleen, thymus, testes, brain, pancreas, lungs, kidneys, peritoneal cavity, subcutaneous tissues, fat pads and other locations [J. Rozga et al., Intraabdominal Organ Transplantation 2000; R. G. Landes Co., USA, 1994: 129]. No one site has emerged as clearly superior to another.

With regard to rejection, encapsulation has been shown to allow transplantation of cells without immunosuppression [F. Lim and A. Sun, “Microencapsulated islets as bioartificial endocrine pancreas,” Science 210:908 (1980); N. Gomez et al., “Evidence for survival and metabolic activity of encapsulated xenogeneic hepatocytes transplanted without immunosuppression in Gunn rats,” Transplantation 63:1718 (1997); V. Dixit et al., Cryopreserved microencapsulated hepatocytes transplantation studies in Gunn rats,” Transplantation 55:616 (1993); and K. Takebe et al., “Xenogeneic (pig to rat) fetal liver fragment transplantation using microcapsules for immuno isolation,” Cell Transplant 5 (suppl. No. 1):S31 (1996)]. In this technique, cells are surrounded by a semipermeable membrane that allows free exchange of oxygen, nutrients, and metabolites while preventing the passage of cells and high molecular weight substances such as immunocytes, antibodies, and complement factors.

A limitation of encapsulated cell therapy is that cell survival depends on nutrient and oxygen availability at the transplantation site. The latter requires neovascularization at the implantation site, a process that requires a significant amount of time. Attempts have been made to induce blood vessel formation through use of a three-dimensional biodegradable matrix, vascular endothelial growth factor, or use of capsules made of polymers inducing local inflammatory response [S. S. Kim et al., “Survival and function of hepatocytes on a novel three-dimensional synthetic biodegradable polymer scaffold with an intrinsic network of channels,” Ann. Surg. 228:8 (1998); M. B. Yang et al., “Hollow fibers for hepatocyte encapsulation and transplantation: studies of survival and function in rats,” Cell Transplant. 3:373 (1994); N. Trivedi et al., “Improved vascularization of planar membrane diffusion devices following continuous infusion of vascular endothelial growth factor,” Cell Transplant. 9:115 (2000); and S. K. Hunter et al., “Promotion of neovascularization around hollow fiber artificial organs using biologically active substances,” Amer. Soc. Art. Int. Organs J. 45:37 (1999)]. None of these techniques has proved to be successful, and no cell transplantation therapy has evolved from them. In any event, even if such techniques could solve the problem of cell survival, they would invariably create another. For example, placing foreign body material such as hollow fibers in the abdominal cavity may cause complications, including development of peritoneal adhesions, abscess formation, ileus, intestinal perforation, to name but a few.

An effective cell transplantation technique should preferably have the following characteristics: (1) it is simple to perform; (2) it results in early cell survival; (3) it provides for transplantation of an adequate number of cells; (4) transplanted cells do not suffer from the effects of nutrient and oxygen deprivation (anoxia/hypoxia), and (5) transplanted cells are able to express specific (that is, desired) functions in vivo. No one has conceived a technique that has all of these characteristics.

Investigators have attempted intraperitoneal infusion of cells [J. Rozga et al. “Repeated Intraportal Hepatocyte Transplantation In Rats”. Cell Transplant. 4: 237-243 (1994); L. Wen et al., “Encapsulated xenogeneic hepatocytes remain functional after peritoneal implantation despite immunization of the host,” J. Hepatol. 29:960 (1998); A. A. Demetriou et al., “Replacement of liver function in rats,” Science 233:1190 (1986); and D. E. Sutherland et al., “Hepatocellular transplantation in acute liver failure,” Surgery 82:124 (1977)]. However, this mode of cell delivery is limited by the need to introduce various types of matrix to provide a substratum for cell attachment and differentiation, as well as a need for rapid neovascularization to provide the grafted cells with oxygen and nutrients. The latter may be difficult to achieve when transplanted cells are enclosed in a semipermeable membrane.

An important need exists in the art, therefore, to provide an encapsulation technique that obviates the above-mentioned limitations.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIG. 1 is executed in color and depicts light microscopic examination of the spleens seeded with allogeneic hepatocytes encapsulated in hollow fibers in accordance with an embodiment of the present invention. After 6 days (FIGS. 1A and 1B) and 28 days (FIGS. 1C and 1D) the cells remained viable. Original magnification 100× (FIGS. 1A and 1C) and 200× (FIGS. 1B and 1D). Arrows point to the surface of hollow fibers.

FIG. 2A is executed in color and depicts light microscopic examination of rat spleens seeded with human hepatoblastoma-derived HepG2 cells encapsulated in alginate/poly-L-lysine capsules in accordance with an embodiment of the present invention. After 4 weeks, HepG2 cells remained viable and, remarkably, no fibrosis developed around the capsule (original magnification 100×). FIG. 2B is executed in color and depicts immunocytochemical staining of encapsulated HepG2 cells transplanted into the rat spleen in accordance with an embodiment of the present invention. After 4 weeks, HepG2 cells stained positive for human albumin (original magnification 100×).

FIG. 3 is executed in color and depicts a detection of human albumin in rat sera in accordance with an embodiment of the present invention. As described below in Table 2, Group A rats were transplanted with encapsulated HepG2 cells, whereas Group B rats received transplantation of free HepG2 cells. Human albumin was detected in Group A rats at all time points studied (i.e., lanes 1 to 6). In Group B rats, human albumin was detected in serum samples obtained on days 1, 3, and 7 (i.e., lanes 7 to 10), but not on day 14 or day 28 (i.e., lanes 11 to 12) after transplantation. Human albumin was not detected in serum samples from Group C (empty fibers; i.e., lane E) or Group D (syngeneic rat hepatocytes; i.e., lane S) rats.

FIG. 4 depicts a measurement of anti-HepG2 antibodies in rat serum samples in accordance with an embodiment of the present invention. In Group B rats, serum levels of anti-HepG2 antibodies increased sharply three days after transplantation, but from day 7 onward returned to the level seen in rats transplanted with empty capsules. In Group A rats, an increase in serum levels of anti-HepG2 antibodies occurred later and was both brief and much less pronounced than in Group B rats. Data (optical density, “OD”) are shown as mean±S.D. One-way analysis of variance: *P<0.05 and **P<0.01.

### DETAILED DESCRIPTION OF THE INVENTION

The present invention is based on the inventors' discovery that, by implanting encapsulated cells in the spleen of a recipient, the cells may survive without suffering immediate/early cell anoxia, inadequate cell nourishment, cell death, or immune rejection—all without additional immunosuppression. Furthermore, oxygen and various nutrients may be provided to the cells without need for artificially prompting additional vascularization. Moreover, as the implantation may be performed percutaneously or endoscopically (e.g., laparoscopically), or by similar minimally invasive means, a recipient need not endure a highly traumatic surgical procedure.

In one embodiment, the method of the invention comprises the steps of providing a cell that is appropriate for implantation, encapsulating the cell in a suitable encapsulation material, and implanting the encapsulated cells in the spleen of a recipient. A wide variety of cells appropriate for use in accordance with the present invention exist, as will be readily appreciated by one of skill in the art of cell implantation. Appropriate cells (autologous, allogeneic, xenogeneic) include, for example, hepatocytes, all types of stem cells, insulin producing cells including cells derived from stem cells of any origin (e.g., pancreatic islet cells, isolated pancreatic beta cells, insulinoma cells, etc.), endocrine hormone-producing cells (e.g., parathyroid, thyroid, adrenal, etc.) and any genetically engineered cells that secrete therapeutic agents, such as proteins or hormones for treating disease or other conditions, and genetically engineered cells that secrete diagnostic agents.

Suitable encapsulation materials include any selectively permeable membrane made of polymer or any other material (e.g., organic, inorganic, biologic), including, for example, polyvinylidene difluoride (PVDF), cellulose diacetate, polysulfone, chitosan, poly-L-lysine, poly-L-lysine-alginate, and silica gel (sol-gel). The encapsulation materials may be formed into any desirable geometric configuration, such as, for example, hollow fibers, spheres, capsules, microcapsules, tubes, tubules, or pouches. The selection of a geometric configuration may depend upon the type of cell being implanted and the encapsulation material being employed. Such a geometric configuration may be selected without undue experimentation by one of skill in the art of cell encapsulation and transplantation, as can an appropriate encapsulation material.

The encapsulated cells of the present invention may be implanted in a recipient by any suitable method. In a preferred embodiment of the present invention, encapsulated cells are implanted percutaneously under ultrasonographic control (guidance). In another embodiment of the present invention, one may implant the cells of the invention under direct visual control using a laparoscope. It will readily be appreciated by one of skill in the art of surgical transplantation of encapsulated cells, however, that other suitable means of implantation are available and may be determined without undue experimentation.

The inventors demonstrate for the first time herein that implantation of encapsulated cells in the spleen results in excellent survival and function of the transplanted cells. While not wishing to be bound by any theory, it is believed that these effects may be attributed to the capsules of the present invention being in direct contact with the blood traversing the splenic pulp. Accordingly, the cells may not suffer from immediate/early oxygen deprivation (anoxia/hypoxia) while receiving adequate nourishment.

A number of encapsulation technologies applicable to living cells have been developed in recent years [F. Lim and A. Sun at 908; Gomez et al. at 1718; Dixit et al. at 616; Takebe et al. at S31; Yang et al. at 373; Wen et al. at 960; Z. H. Cai et al., “Development and evaluation of a system of microencapsulation of primary rat hepatocytes,” Hepatol. 10:855 (1989); and D. W. Scharp et al., “Protection of encapsulated human islets implanted without immunosuppression in patients with type I or type II diabetes and in nondiabetic control subject,” Diabetes 43:1167 (1994)]. In such systems, cells are surrounded by a semipermeable membrane which should ideally allow free exchange of oxygen, nutrients, and metabolites while excluding the passage of cells and high molecular-weight substances such as immunocytes, antibodies, and complement factors. The present invention demonstrates that encapsulated cell technologies allow transplantation without immunosuppression.

Remarkably, even though the spleen as a transplantation site is not immunologically privileged, survival and function of encapsulated allogeneic and xenogeneic cells were excellent, as described in the Examples below. Moreover, there was no fibrosis around the alginate/poly-L-lysine (ALP) capsules containing human HepG2 cells after implantation in rat spleens. However, though a thin layer of fibroblasts surrounded the PVDF fibers containing allogeneic hepatocytes, this resulted from foreign body reaction rather than immune-mediated response, as judged by the presence of a similar fibrotic layer around empty fibers. Although the latter finding stresses the importance of developing hollow fibers with improved biocompatibility, it is worth noting that an approximately 20μ-thick layer of fibroblasts surrounding the fiber wall did not deprive the cells inside the fiber of either nutrients or oxygen. This shows that even the PVDF fiber may be considered for intrasplenic cell therapy.

Intrasplenic encapsulated cell therapy offers numerous advantages. First, encapsulated cells are separated from the bloodstream by a semipermeable membrane only. Second, by using membranes with a molecular weight cut-off around 100 kDa, mismatched cells (e.g., allogeneic, xenogeneic) are protected against rejection. Third, encapsulation prevents the migration of cells to the liver, a phenomenon known to occur after the intrasplenic transplantation of free (i.e., non-encapsulated) cells [S. Gupta et al., “Permanent engraftment and function of hepatocytes delivered to the liver: implication for gene therapy and liver repopulation,” Hepatol. 14:144 (1991); and J. P. Vroemen et al., “Hepatocyte transplantation for enzyme deficiency disease in congeneic rats,” Transplant. 42:130 (1986)]. Fourth, transplantation of encapsulated cells may be performed percutaneously or through a minimally invasive laparoscopic approach [R. J. Rosenthal et al., “Techniques for intrasplenic hepatocytes transplantation in the large animal model,” Surg. Endos. 10:1075 (1996)]. Fifth, the spleen is a well-defined parenchymal organ and the fate of transplanted cells (e.g., tagged cells) could be studied using an array of modern diagnostic techniques. Sixth, and finally, by employing the present invention for the implantation of pancreatic islets or any other insulin producing cells, including those derived from stem cells of any origin, insulin produced thereby may be delivered directly to the liver (i.e. in a physiological manner).

### EXAMPLES

The Examples discussed herein demonstrate that intrasplenic implantation of encapsulated cells may allow introduction of allogeneic or xenogeneic cells to a mammal and long-term survival and function of the transplanted cells without the need for immunosuppression. All experiments described in this section were performed following protocols approved by the Institutional Animal Care and Use Committee and in accordance with National Research Council guidelines for humane care of experimental animals.

### Example 1

### Preparation of Animals and Cells

Adult male Sprague-Dawley (SD) and inbred Lewis rats (200 g-300 g) were obtained from Harlan Sprague-Dawley, Inc. (San Diego, Calif.). Animals were housed in a climate-controlled-(21° C.) room with a 12-hour light-dark cycle, and were given tap water and standard laboratory rat chow (Rodent Chow 5001; obtained from Ralston Purina, St. Louis, Mo.) ad libitum. All operations were performed under general (metaphane) anesthesia using sterile surgical technique.

Rat liver cells were harvested and enriched according to methods well known in the art. Cells were first harvested by in situ two-step ethylenediamine tetraacetic acid (EDTA)/collagenase digestion (both obtained from Sigma Chemical Co., St. Louis, Mo., hereinafter “Sigma”), as described by M. Holzman et al. “Selective intraportal hepatocyte transplantation in analbuminemic and Gunn rats. Transplantation 55: 1213-1219 (1993). After enrichment through a Percoll (obtained from Pharmacia Biotechnology Co., Piscataway, N.J.) density gradient, cell viability was greater than 95%, as judged by trypan blue exclusion test.

HepG2 cells were used in most experiments, because this human hepatoma-derived cell line is well characterized, secretes albumin, and is easy to maintain in culture. HepG2 cells (obtained from American Type Culture Collection, “ATCC”; Manassas, Va.) were cultured in Minimum Essential Eagle's Medium (obtained from ATCC) supplemented with 10% fetal bovine serum (“FBS,” obtained from Sigma) and antibiotics (penicillin G 10,000 units/ml, streptomycin 10,000 μg/ml, amphotericin B 25 μg/ml; obtained from Omega Scientific, Inc., Tarzana, Calif., hereinafter “Omega”). The culture medium was changed every other day. At confluence, cells were detached using 0.25% trypsin/EDTA (obtained from Sigma) and tested for viability; more than 95% of cells excluded trypan blue.

### Example 2

### Preparation of Capsules

PVDF hollow fibers with a 100 kDa molecular weight cut-off were a gift from Spectrum Laboratories, Inc. (Rancho Dominguez, Calif.). Hollow fibers were soaked in deionized water for 30 minutes at room temperature and pre-wet for 30 minutes in 100% ethyl alcohol. After rinsing in saline to remove alcohol, 5×10⁵ rat hepatocytes were suspended in Dulbecco's modified Eagle medium (DMEM)/5% FBS (obtained from Sigma) and loaded into a 4-cm-long hollow fiber using a 22 gauge Venocath (obtained from Becton Dickinson Vascular Access, Sandy, Utah). The two fiber ends were sealed with 6-0 silk ligatures (obtained from Ethicon, Inc., Somerville, N.J.) and the fiber was either maintained in culture in a hormonally-defined DMEM or immediately implanted in a rat spleen.

### Example 3

### Encapsulation of Cells in ALP Capsules

Encapsulation of cells was carried out using the syringe extrusion technique. Briefly, either 1×10⁸ HepG2 cells or 1×10⁸ primary rat hepatocytes were suspended in 10 ml of 0.9% sodium chloride, which contained 1.5% sodium alginate (obtained from Sigma) and 0.045% (w/v) rat tail collagen type I (obtained from Collaborative Biomedical Product, Bedford, Mass.), and antibiotics 1:100 (see Example 1 for antibiotics). Droplets of this suspension gelled upon collection in a 1.1% CaCl₂ solution.

Following washing in 0.1% 2-N-cyclohexylaminoethane sulfonic acid (“CHES,” obtained from Sigma), 1.1% CaCl₂ and then twice in 0.9% NaCl, the microspheres were coated with 0.05% (w/v) poly-L-lysine (obtained from Sigma) for 10 minutes and washed with 0.1% CHES, 1.1% CaCl₂ and 0.9% NaCl. Exposure for 4 minutes to 0.15% sodium alginate (obtained from Sigma) formed the outer layer of the membrane. The droplets were then washed twice in normal saline. The microcapsules measured from about 800μ to about 1000μ in diameter, and were immediately transplanted.

### Example 4

### Hepatocyte Transplantation

A small left subcostal incision was made in the animals and the spleen was exposed. Using a 14 gauge Venocath as the “deployment device,” a 4-cm long PVDF fiber (empty or loaded with 5×10⁵ hepatocytes) was inserted into the spleen through the lower pole along the spleen axis. After implantation, the entry site was closed with a single 4-0 silk ligature to prevent bleeding. Experiments were performed as summarized in Table 1.

TABLE 1
Summary of experiments

| | |
|---|---|
| Group I | SD rats (n = 15) received intrasplenic transplantation of |
| | 5 × 10⁵ allogeneic SD rat hepatocytes seeded in a |
| | PVDF fiber |
| Group II | SD rats (n = 15) received intrasplenic transplantation of free |
| | 5 × 10⁵ allogeneic SD rat hepatocytes |
| Group III | SD rats (n = 15) received intrasplenic |
| | implantation of an empty PVDF fiber |

Rats from each group were killed in batches of three to five after 2, 4, 6, 14, and 28 days. At sacrifice, spleens were removed and fixed in 10% buffered formalin. Light microscopic examination was performed on paraffin-embedded 5μ-thick sections stained with hematoxylin and eosin.

### Example 5

### HepG2 Cell Transplantation

Inbred Lewis rats were used as transplant recipients. Approximately 1.0×10⁷ microencapsulated HepG2 cells suspended in 1 mL of physiologic saline were injected into the spleen using a 14 gauge Venocath (obtained from Becton Dickinson Vascular Access, Sandy, Utah). Hemostasis at the injection site was achieved with a 4-0 silk ligature. There were four experimental groups, as described in Table 2.

TABLE 2
Experimental groups

| | |
|---|---|
| Group A | Rats (n = 35) received intrasplenic transplantation of |
| | encapsulated HepG2 cells |
| Group B | Rats (n = 35) received intrasplenic transplantation of |
| | free HepG2 cells |
| Group C | Rats (n = 35) received intrasplenic transplantation of |
| | empty microcapsules |
| Group D | Rats (n = 35) received intrasplenic transplantation of |
| | 1.0 × 10⁷ microencapsulated syngeneic rat hepatocytes |
| | (i.e., to determine the survival of intrasplenically seeded cells |
| | when no immune reaction was expected to develop around the |
| | capsule wall) |

Rats from each group were killed in batches of 5 rats each at 0, 1, 3, 5, 7,14, and 28 days post-transplantation. At sacrifice, blood was collected by aortic puncture and the spleen was removed and processed for morphologic examination.

### Example 6

### Detection of Human Albumin in Rat Serum

Western blot analysis was utilized to detect the human albumin. Briefly, sodium dodecylsulfate-polyacrylamide gel electrophoresis was performed in a vertical tank (Mini-Protein II Electrophoresis cell, obtained from Bio-Rad). Rat serum (diluted 1:10,000 in PBS, obtained from Sigma) was diluted 1:2 in Laemmli sample buffer (obtained from Bio-Rad) and denatured by heating for 3 minutes at 100° C. 8 μL of this solution was deposited on a 5% stacking gel and a 12% separating gel. Migration was run at 20 mA for 15 minutes and then at 40 mA for 1 hour. Reference proteins (obtained from Bio-Rad) were run simultaneously in a control lane.

Separated proteins were transferred onto a nitrocellulose membrane in Semi-Dry Transfer Cell (obtained from Bio-Rad), which was then saturated with 5% non-fat milk in PBS for 2 hours at room temperature and 1:500 rabbit anti-human albumin antibody (obtained from DAKO Co., Carpinteria, Calif., hereinafter “DAKO”) was applied at 4° C. overnight. After three washings with PBS-T, a peroxidase-conjugated goat anti-rabbit antibody (obtained from Santa Cruz Biotechnology Inc.) was applied for 30 minutes at room temperature. Western blotting detection reagents (i.e., ECL; obtained from Amersham Pharmacia Biotech, Inc., Piscataway, N.J.) revealed the reaction.

### Example 7

### Detection of Anti-HepG2 Antibodies in Rat Serum

Anti-HepG2 antibodies were detected by ELISA. In brief, HepG2 cells were cultured in 96-well microtiter plates (obtained from Corning Coaster Corporation, Corning, N.Y.) for 6 hours in Leibovitz's L-15 Medium (obtained from GIBCO Invitrogen Corporation, Carlsbad, Calif.) and then fixed with 0.1% glutaraldehyde (obtained from Sigma) for 5 minutes. The cells were placed at 4° C. in SuperBlock blocking buffer in PBS (obtained from Pierce) overnight. After washing three times with PBS, rat sera diluted 1:40 in PBS were added for 2 hours at room temperature. All samples were analyzed in triplicate.

After washing three times with PBS, 1:1000 goat anti-rat IgG labeled with horseradish peroxidase (obtained from Vector Laboratories, Inc., Burlingame, Calif., hereinafter “Vector”) was added for 1 hour at room temperature. ABTS (obtained from Roche) was used to reveal peroxidase activity. In negative controls, rat serum was omitted. Plates were read on an automated Microplate Reader 3550-UV (obtained from Bio-Rad).

### Example 8

### Morphological Evaluation

The specimens were fixed in 10% formalin, dehydrated, and embedded in paraffin. Sections 7μ-thick were stained with hematoxylin-eosin. Additionally, human albumin was detected in HepG2 cell implants using standard immunohistochemical technique. Rabbit anti-human albumin antibody (obtained from DAKO) was used as the primary antibody and anti-rabbit biotin conjugated IgG (obtained from Vector) was used as the secondary antibody. The slides were developed with diaminobenzine.

### Example 9

### Intrasplenic Transplantation of Hepatocytes in PVDF Fibers

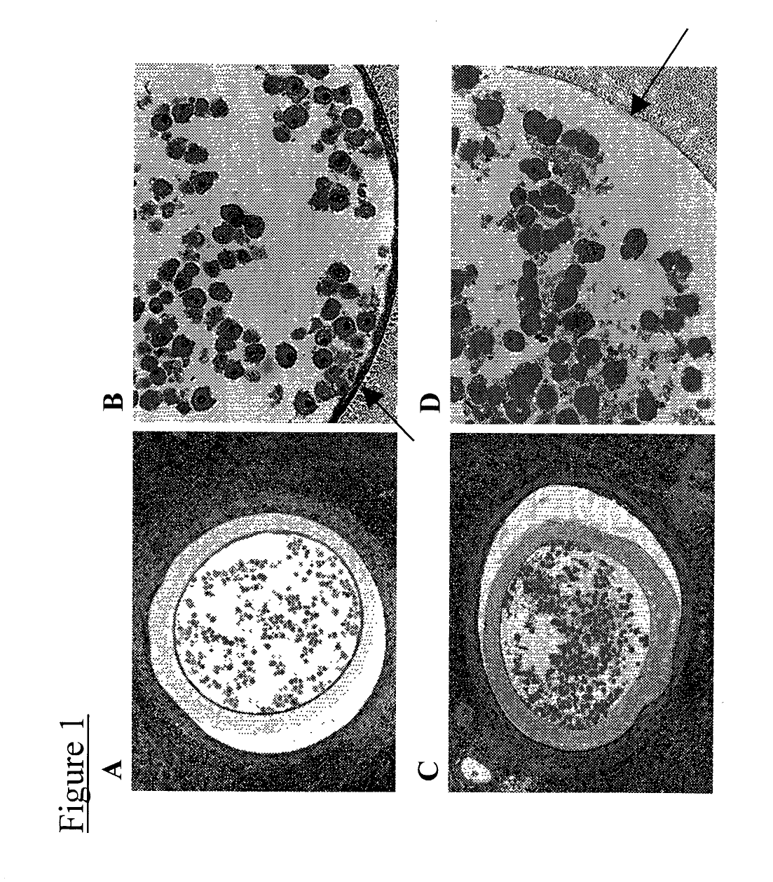

Empty fibers produced only mild fibrotic reaction. The presence of allogeneic cells in the fibers did not aggravate per-fiber response (FIGS. 1A-1D). Moreover, Group I cells transplanted in 100 kDa fibers remained viable throughout the study period, while Group II free hepatocyte transplants survived in the spleen for 6 days only.

### Example 10

### Intrasplenic Transplantation of Cells in ALP Capsules

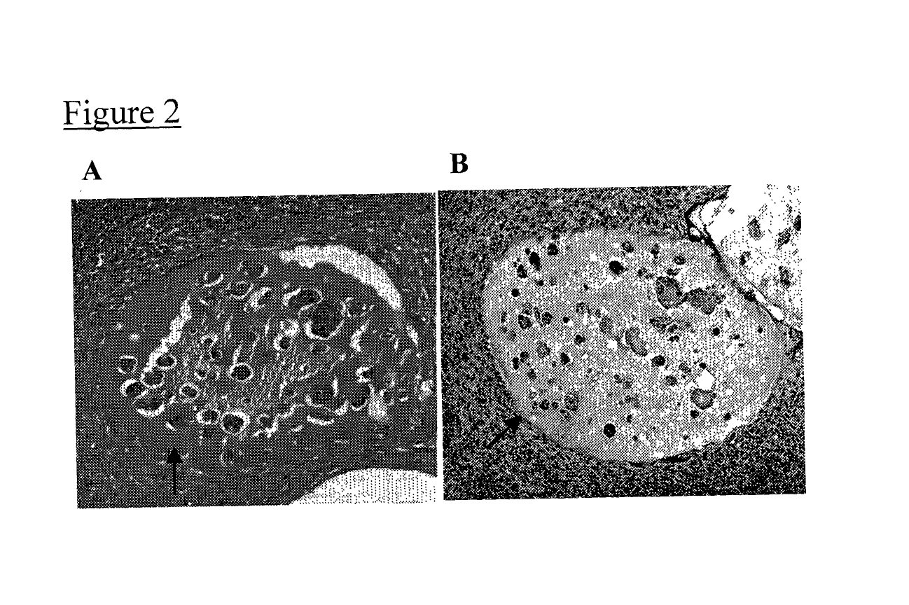

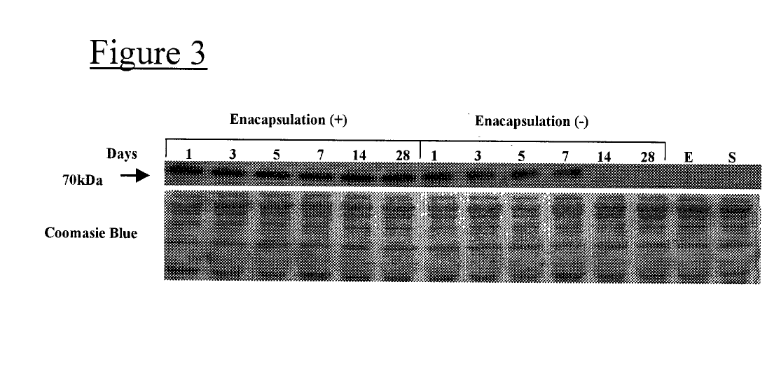

In Group A rats, ALP capsules contained viable HepG2 cells at all time points studied and no peri-capsular fibrosis was noted (FIG. 2A). The cells stained positive for human albumin (FIG. 2B) which was also detected in all blood samples by Western blotting (FIG. 3).

In Group B rats, viable HepG2 cells could be found in the spleens for 7 days only. Additionally, human albumin was detected only in blood samples obtained at 1, 3, and 7 days after transplantation (FIG. 3).

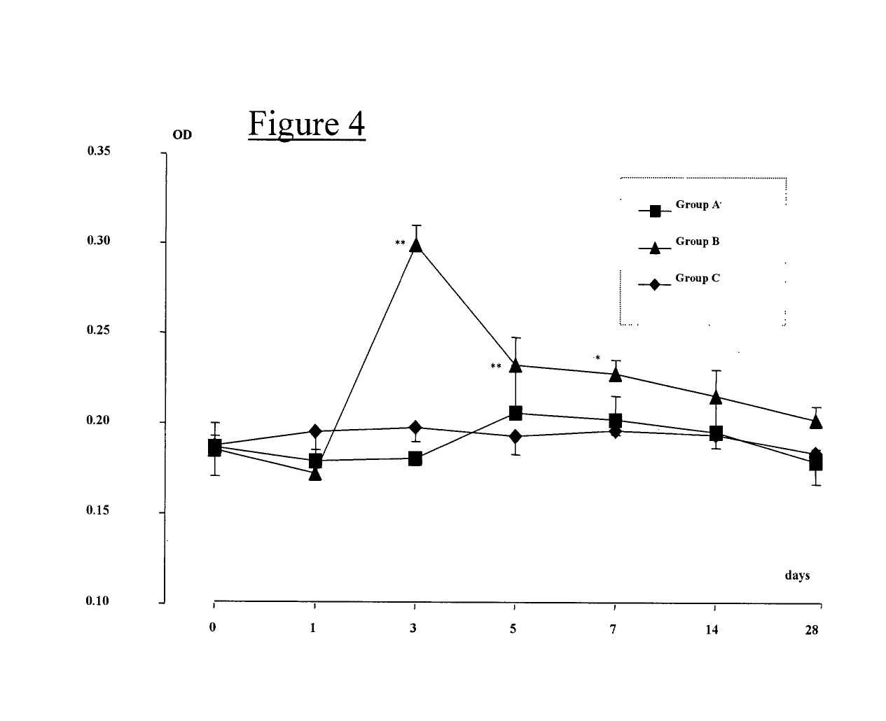

After transplantation of free HepG2 cells, serum levels of anti-HepG2 antibodies increased sharply at 3 days after transplantation but from day 7 onward returned to the level seen in rats transplanted with empty capsules (FIG. 4). In Group A rats, an increase in serum levels of anti-HepG2 antibodies occurred later and was both brief and much less pronounced than in Group B rats (FIG. 4).

Group D rats transplanted with microencapsulated syngeneic rat hepatocytes showed excellent cell survival and no pericapsular fibrosis.

While the description above refers to particular embodiments of the present invention, it will be understood that many modifications may be made without departing from the spirit thereof. The accompanying claims are intended to cover such modifications as would fall within the true scope and spirit of the present invention. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

What is claimed is:

1. A method for implanting encapsulated cells in a recipient, the method comprising the step of implanting encapsulated cells in the recipient's spleen.

2. The method of claim 1, wherein the encapsulated cells are selected from the group consisting of encapsulated autologous cells, encapsulated allogeneic cells, and encapsulated xenogeneic cells.

3. The method of claim 2, wherein the encapsulated cells are encapsulated stem cells.

4. The method of claim 2, wherein the encapsulated cells are encapsulated insulin producing cells of any origin.

5. The method of claim 4, wherein the encapsulated cells are selected from the group consisting of encapsulated pancreatic islet cells, encapsulated pancreatic beta cells, and encapsulated insulinoma cells.

6. The method of claim 2, wherein the encapsulated cells are encapsulated, endocrine hormone-producing cells

7. The method of claim 6, wherein the encapsulated cells are selected from the group consisting of encapsulated parathyroid cells, encapsulated thyroid cells, encapsulated adrenal cells, and encapsulated pituitary cells.

8. The method of claim 2, wherein the encapsulated cells are selected from the group consisting of encapsulated cells that secrete therapeutic agents and encapsulated cells that secrete diagnostic agents.

9. The method of claim 1, wherein the encapsulated cells comprise cells encapsulated within a semipermeable membrane.

10. The method of claim 9, wherein the semipermeable membrane comprises a material selected from the group consisting of alginate/poly-L-lysine and hollow fibers.

11. The method of claim 10, wherein the hollow fibers further comprise polyvinylidene difluoride (PVDF) with or without surface modification enhancing bio- and hemo-compatibility of the semipermeable membrane.

12. The method of claim 1, wherein the method of implanting the encapsulated cells further comprises implanting the encapsulated cells percutaneously under the control of an imaging technique selected from the group consisting of ultrasonography, fluoroscopy, magnetic resonance imaging, endoscopic imaging, and laparoscopic imaging.

13. The method of claim 1, wherein following the step of implanting the encapsulated cells, the cells survive in the recipient with reduced immunosuppression or without immunosuppression.

14. The method of claim 1, wherein following the step of implanting the encapsulated cells, the encapsulated cells remain located in the recipient's spleen.

15. A method for implanting cells in a recipient, the method comprising the steps of:
providing cells;
encapsulating the cells in an encapsulation material; and
implanting the cells in the spleen of a recipient.

16. The method of claim 15, wherein the cells are selected from the group consisting of autologous cells, allogeneic cells, and xenogeneic cells.

17. The method of claim 16, wherein the cells are stem cells.

18. The method of claim 16, wherein the cells are insulin producing cells of any origin.

19. The method of claim 18, wherein the cells are selected from the group consisting of pancreatic islet cells, pancreatic beta cells, and insulinoma cells.

20. The method of claim 16, wherein the cells are endocrine hormone-producing cells.

21. The method of claim 20, wherein the cells are selected from the group consisting of parathyroid cells, thyroid cells, adrenal cells, and pituitary cells.

22. The method of claim 16, wherein the cells are selected from the group consisting of cells that secrete therapeutic agents and cells that secrete diagnostic agents.

23. The method of claim 15, wherein the step of encapsulating the cells in an encapsulation material further comprises encapsulating the cells within a semipermeable membrane.

24. The method of claim 23, wherein the semipermeable membrane comprises a material selected from the group consisting of alginate/poly-L-lysine and hollow fibers.

25. The method of claim 24, wherein the hollow fibers further comprise polyvinylidene difluoride (PVDF) with or without surface modification enhancing bio- and hemo-compatibility of the semipermeable membrane.

26. The method of claim 15, wherein the method for implanting cells further comprises implanting the cells percutaneously under the control of an imaging technique selected from the group consisting of ultrasonography, fluoroscopy, magnetic resonance imaging, endoscopic imaging, and laparoscopic imaging.

27. The method of claim 15, wherein following the step of implanting the cells, the cells survive in the recipient with reduced immunosuppression or without immunosuppression.

28. The method of claim 15, wherein following the step of implanting the cells, the cells remain located in the spleen of the recipient.

29. A method for implanting cells in a recipient, the method comprising the steps of:
providing cells;
encapsulating the cells in a capsule; and
implanting the capsule in the spleen of a recipient.

30. The method of claim 29, wherein the cells are selected from the group consisting of autologous cells, allogeneic cells, and xenogeneic cells.

31. The method of claim 30, wherein the cells are stem cells.

32. The method of claim 30, wherein the cells are insulin producing cells of any origin.

33. The method of claim 32, wherein the cells are selected from the group consisting of pancreatic islet cells, pancreatic beta cells, and insulinoma cells.

34. The method of claim 30, wherein the cells are endocrine hormone-producing cells.

35. The method of claim 34, wherein the cells are selected from the group consisting of parathyroid cells, thyroid cells, adrenal cells, and pituitary cells.

36. The method of claim 30, wherein the cells are selected from the group consisting of cells that secrete therapeutic agents and cells that secrete diagnostic agents.

37. The method of claim 29, wherein the capsule further comprises a semipermeable membrane.

38. The method of claim 37, wherein the semipermeable membrane comprises a material selected from the group consisting of alginate/poly-L-lysine and hollow fibers.

39. The method of claim 38, wherein the hollow fibers further comprise polyvinylidene difluoride (PVDF) with or without surface modification enhancing bio- and hemo-compatibility of the semipermeable membrane.

40. The method of claim 29, wherein the method for implanting cells further comprises implanting the capsule percutaneously under the control of an imaging technique selected from the group consisting of ultrasonography, fluoroscopy, magnetic resonance imaging, endoscopic imaging, and laparoscopic imaging.

41. The method of claim 29, wherein following the step of implanting the capsule, the cells survive in the recipient with reduced immunosuppression or without immunosuppression.

42. The method of claim 29, wherein following the step of implanting the capsule, the cells remain located in the spleen of the recipient.
